(19) United States
(12) Patent Application Publication
(10) Pub. No.: US 2026/0151463 A1
(43) Pub. Date: Jun. 4, 2026

(54) COMPOSITIONS AND METHODS FOR TREATING PARKINSON'S DISEASE

(71) Applicants: UNM Rainforest Innovations, Albuquerque, NM (US); United States Department of Veterans Affairs, Washington, DC (US)

(72) Inventors: Henry C. LIN, Albuquerque, NM (US); Sudha B. SINGH, Albuquerque, NM (US)

(21) Appl. No.: 19/409,073

(22) Filed: Dec. 4, 2025

Related U.S. Application Data
(60) Provisional application No. 63/727,971, filed on Dec. 4, 2024.

(51) Int. Cl. A61K 38/46; A61K 31/196; A61K 31/198; A61K 31/353; A61K 31/436; A61K 38/13

(52) U.S. Cl. CPC A61K 38/465; A61K 31/196; A61K 31/198; A61K 31/353; A61K 31/436; A61K 38/13; C12Y 301/03001

(57) ABSTRACT

This disclosure describes compositions and methods for treating Parkinson's Disease by targeting production and secretion of α-synuclein. This disclosure identifies mechanisms by which α-synuclein aggregates form and provide compositions and methods of treatment that inhibit and/or ameliorate the effects of α-synuclein aggregates in Parkinson's Disease.

### CROSS-REFERENCE TO RELATED APPLICATION

This application claims the benefit of U.S. Provisional Patent Application No. 63/727,971, filed Dec. 4, 2024, which is incorporated herein by reference in its entirety.

### SUMMARY

This disclosure describes compositions and methods for treating Parkinson's Disease by targeting production and secretion of α-synuclein.

In one or more embodiments, the compositions and methods target gut dysbiosis characterized by bloom of sulfate reducing bacteria.

In one or more embodiments, the compositions and methods suppress luminal growth of sulfate reducing bacteria and/or binding of its metabolite hydrogen sulfide.

In one or more embodiments, the compositions and methods target a TLR2 signaling pathway activated by sulfate reducing bacteria.

In one or more embodiments, the compositions and methods increase proteasomal or lysosomal destruction of intracellular sulfate reducing bacteria.

In one or more embodiments, the compositions and methods inhibit synuclein aggregation.

In one or more embodiments, the compositions and methods inhibit secretion of synuclein or other sulfate-reducing-bacteria-induced factors from intestinal cells.

In one or more embodiments, the compositions and methods inhibit redistribution of intracellular dopamine (DA) from synaptic vesicles into the cytosol or that increase VMAT2 protein to facilitate uptake of cytosolic DA into synaptic vesicles.

In one or more embodiments, the compositions and methods interfere with sulfate reducing bacteria-induced ER stress.

In one or more embodiments, the compositions and methods interfere with sulfate reducing bacteria-induced proinflammatory response.

In one or more embodiments, the compositions and methods inhibit sulfate reducing bacteria-induced nitric oxide synthase.

In one or more embodiments, the compositions and methods interfere with inhibition of tyrosine hydroxylase (TH) expression or that increase tyrosine hydroxylase expression.

In one or more embodiments, the compositions and methods inhibit binding of synuclein to TH.

In one or more embodiments, the compositions and methods modulate Gal-1 and/or Gal-3.

In one or more embodiments, the compositions and methods overcome DSV effects and enhance phagocytosis and clearance of synuclein aggregates by microglia.

In one or more embodiments, the compositions and methods reduce DSV-induced chronic astrogliosis.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. DSV induces increase in synuclein aggregate in intestinal enteroendocrine STC-1 cells. Cells were infected for 24 hours with various DSV MOIs. Cells were then fixed and processed for immunofluorescence using anti-α-synuclein aggregate antibody. Percentage of cells positive for aggregates were counted and values were compared to control (uninfected) cells. Data represent mean±SEM. A two-tailed students t-test was used to analyze the statistical difference between control and DSV-infected cells. P values<0.05 were considered significant. **P<0.01, *P<0.05.

FIG. 2. DSV induces increase in synuclein protein expression in intestinal enteroendocrine STC-1 cells. Cells were infected for 24 or 48 hours with DSV. Cells were then harvested for western blotting to probe for synuclein expression. Actin was used as loading control. Data represent mean±SEM. Values were normalized to control. A two-tailed student t-test was used to analyze the statistical difference between control and DSV-infected cells. P values<0.05 were considered significant. **P<0.01.

FIG. 3. DSV induces increased extracellular synuclein from intestinal enteroendocrine. STC-1. Cells were infected for 24 hours with various DSV MOIs. Culture supernatants were harvested and amount of extracellular synuclein was measured using ELISA kit. Data represent mean±SEM. Values were normalized against control uninfected cell supernatants. Two-tailed students t-test was used to analyze the statistical difference between the supernatants from control and DSV-infected cells. P values<0.05 were considered significant. **P<0.01, ***P<0.001.

FIG. 4. Sup from DSV-infected STC-1 induce increase in synuclein aggregate in neuronal Sy5y cells. STC-1 cells were infected for 24 hours with various DSV MOIs. Culture supernatants (sup) were through 0.2 μm filters and added to Sy5y cells, for 24 hours. Sy5y cells were then fixed and processed for immunofluorescence using anti-α-synuclein aggregate antibody. Percentage of cells positive for aggregates were counted and values were compared to cells treated with control uninfected sup from STC-1 cells. Data represent mean±SEM. A two-tailed students t-test was used to analyze the statistical difference between the groups. P values<0.05 were considered significant. *P<0.05.

FIG. 5. FIG. 5. DSV directly induces increase in synuclein aggregate in Sy5y neuronal cells. Cells were infected for 24 hours with various DSV MOIs. Cells were fixed and processed for immunofluorescence using anti-α-synuclein aggregate antibody. Percentage of cells positive for aggregates were counted and values were compared to control uninfected cells. Data represent mean±SEM. A two-tailed students t-test was used to analyze the statistical difference between control and DSV-infected cells. P values<0.05 were considered significant. ***P<0.001 **P<0.01, *P<0.05.

FIG. 6. DSV inhibits tyrosine hydroxylase (TH) protein expression in neuronal Sy5y cells. Cells were infected for 24 hours with DSV at various MOIs. Cells were harvested for western blotting to probe for TH expression. Data represent mean±SEM. A two-tailed student t-test was used to analyze the statistical difference between control and DSV-infected cells. P values<0.05 were considered significant. ***P<0.01, *P<0.05.

FIG. 7. D. piger (D.p) inhibits tyrosine hydroxylase (TH) protein expression in neuronal Sy5y cells. Cells were infected for 24 hours with DSV at various MOIS. Cells were harvested for western blotting to probe for TH expression. Data represent mean±SEM. A two-tailed student t-test was used to analyze the statistical difference between control and DSV-infected cells. P values<0.05 were considered significant. ***P<0.01, *P<0.05.

FIG. 8. DSV and D. piger inhibit extracellular and intracellular dopamine concentration. Sy5y cells were infected for 24 hours with various DSV and D. piger MOIS. Culture supernatants and cells were harvested and amount of extracellular as well as intracellular dopamine was measured using a double sandwich ELISA kit. O.D values were measured at 450 nm.

FIG. 9. Inhibition of DSV-induced synuclein aggregates by various inhibitors. STC-1 cells grown on coverslips were treated with either 100 μM EGCG, 100 μM quercetin, or 100 nM rapamycin for three hours prior to adding DSV at MOI 40 for 48 hours. DMSO vehicle control was added to uninfected control cells and DSV alone-treated cells. Cells were then fixed and processed for immunofluorescence using anti-α-synuclein aggregate antibody. Percentage of cells positive for aggregates were compared to control (uninfected) cells.

FIG. 10. Inhibition of DSV-induced synuclein aggregates by various inhibitors. STC-1 cells grown on coverslips were treated with either 100 μM EGCG, 100 μM Quercetin, or 200 μM C29 three hours prior to adding DSV at MOI 60 for 24 hours. Cells were then fixed and processed for immunofluorescence using anti-α-synuclein aggregate antibody. Percentage of cells positive for aggregates were compared to control (uninfected) cells.

FIG. 11. IAP inhibits DSV-induced synuclein aggregates in intestinal cells. STC-1 cells grown on coverslips were treated with IAP for 24 hours prior to adding DSV at MOI 40 for 48 hours. Cells were then fixed and processed for immunofluorescence using anti-α-synuclein aggregate antibody. Percentage of cells positive for aggregates were compared to control (uninfected) cells.

FIG. 12. IAP inhibits DSV-mediated inhibition of tyrosine hydroxylase (TH) protein expression in Sy5y neuronal cells. Cells were treated with IAP 500 U/ml for 24 hours prior to adding DSV for 24 hours. Cells were harvested for western blotting and probed for TH expression. Actin was used as a loading control. Values were normalized to control.

FIG. 13. DSV induces an increase in synuclein protein expression in Sy5y cells. Cells were infected for 24 hours with DSV at MOI 40 or MOI 60. Cells were then harvested for Western blotting to probe for synuclein expression. Actin was used as loading control. (A) Western blot. (B) Bar graph quantifying data in (A) using Image J software. Data represent mean±SEM. Values were normalized to control.

FIG. 14. DSV induces an increase in TNF-α and mature IL-1β expression in HMC3 cells. Cells were infected for 30 minutes or four hours with DSV at MOI 80. Cells were then harvested and lysed. (A) Protein lysates were run on SDS-PAGE for Western blotting using antibodies specific for TNF-α and IL-1β. Actin was used as a loading control. (B) TNF-α blot was quantified using Image J software. (C) IL-1β blot was quantified using Image J software. Data represent mean±SEM. Values were normalized to control. A two-tailed students t-test was used to analyze the statistical difference between control and DSV-infected cells. P values<0.05 were considered significant. **P<0.01, *P<0.05.

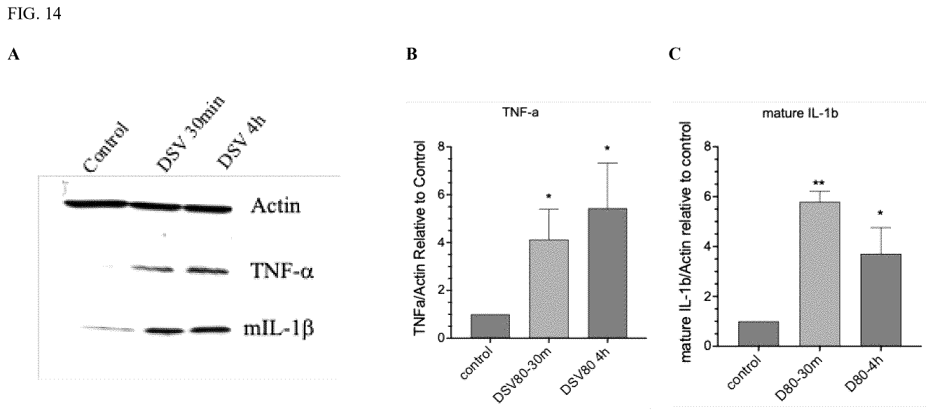

FIG. 15. DSV induces an increase in phosphor-STAT3 expression in HMC3 cells. Cells were infected for 30 minutes or four hours with DSV at MOI 80. Cells were then harvested and lysed. (A) Protein lysates were run on SDS-PAGE for Western blotting using antibodies specific for phosphorylated STAT3 and total STAT3. (B) Ratio of p-STAT3 and total STAT3 were analyzed and normalized to control values. Blot were quantified using Image J software. Data represent mean±SEM.

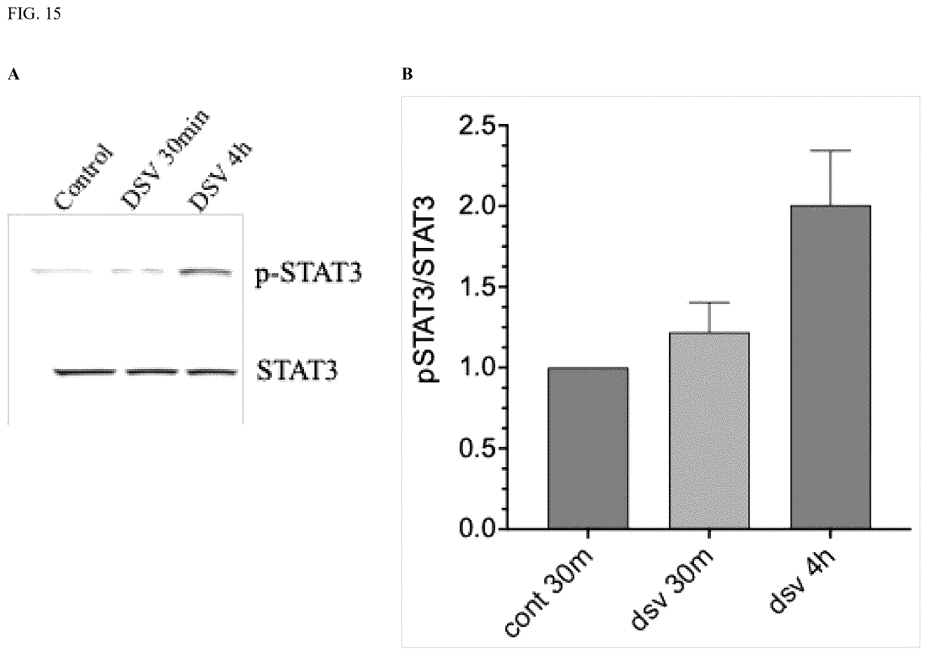

FIG. 16. DSV induces an increase in IL-6 and IL-8 expression in HMC3 cells in time-dependent manner. Cells were infected for various times indicated in (A) with DSV at MOI 80. Cells were then harvested and lysed. (A) Protein lysates were run on SDS-PAGE for Western blotting using antibodies specific for IL-6 and IL-8. Actin was used as a loading control. (B) IL-6 blot was quantified using Image J software. (C) IL-8 blot was quantified using Image J software. Values were normalized to control.

FIG. 17. Supernatant from DSV-infected STC-1 cells induce an increase in mature or cleaved IL-1β in HMC3. STC-1 cells were infected for 24 hours with DSV at MOI 80. Culture supernatants (sup) were through 0.2-μm filters and added to HMC3 cells for 30 minutes. Cells were then harvested and lysed. (A) Protein lysates were run on SDS-PAGE for Western blotting using antibodies specific for IL-1β. Actin was used as a loading control. (B) Blot was quantified using Image J software. Data represent mean±SEM. Values were normalized to control.

FIG. 18 DSV causes an endoplasmic reticulum (ER) stress response in RAW264.7 macrophages. Cells were infected with DSV (MOI20) for various times as indicated. Cells were harvested and lysed. Protein lysates were run on SDS-PAGE for Western blotting using antibodies specific for ER stress response proteins ATF4, ATF6, Xbps1, and Chop1. Actin was used as a loading control.

### DETAILED DESCRIPTION OF ILLUSTRATIVE EMBODIMENTS

Parkinson's disease (PD) is an aging-related neurodegenerative disease. PD is characterized by the progressive loss of dopaminergic neurons and by the presence of Lewy Bodies in the Substantia Nigra pars compacta in PD patients. Tyrosine hydroxylase (TH) catalyzes the conversion of L-tyrosine to L-3,4-dihydroxyphenylalanine (L-DOPA) and is the initial and rate-limiting step in the biosynthesis of dopamine. PD patients are found to have decreased TH in their brain (PMID: 35460433; PMID: 32471089; PMID: 19396395). Lewy bodies are composed of intracellular aggregates of the protein α-synuclein (PMID: 9278044)). Thus, the term synucleinopathies is used to describe those neurodegenerative disorders that involve α-synuclein (α-syn) aggregates into Lewy Bodies. While PD is a neurodegenerative disease, several studies have suggested that this disease may originate in the gut (PMID: 33649989, PMID: 37305758, PMID: 33860738). For example, injecting synuclein fibrils in the gut caused formation of Lewy Bodies in the brain of mouse models (PMID: 31255487). However, in mice that underwent vagotomy, Lewy bodies were not observed in the brain, implying that synuclein aggregates travelled to the brain from the gut via the vagus nerve. PD is also associated with gut microbial dysbiosis (PMID: 33968794), suggesting that gut microbes may play a central role in the development of PD. Further, bacterial amyloid proteins such as E. coli Curli protein can cause aggregation of synuclein both in vivo and in vitro (PMID: 32043464).

Sulfate Reducing Bacteria (SRB) are resident members of human gut that are present as minor members in the colon. Overgrowth of SRB belonging to the genus Desulfovibrio are present in Parkinson's Disease (PD) patients (PMID: 34012926), suggesting that gut microbial dysbiosis is involved in the pathophysiology of PD. In addition, Desulfovibrio species isolated from PD patients, but not those from control subjects (spouses of PD patients), increased synuclein aggregates in the head region when fed to the worm C. elegans (PMID: 37197200). Whether the bacteria induce aggregates of α-synuclein in the gut (which is then transported to the brain via the vagus nerve or bloodstream) and/or directly migrated to the brain by translocation of bacteria or their secreted products through leaky gut into the circulation remains unknown. The relationship between Desulfovibrio spp. and the accumulation and aggregation of α-synuclein forming Lewy Bodies is unclear. An in vitro study was performed to investigate the underlying mechanisms of how Desulfovibrio may contribute in the development of PD by using intestinal cells and neuronal cells.

### Effect of Desulfovibrio on Synuclein Aggregation and Expression

Alpha-synuclein (α-syn) is an ˜18 kDa protein found in the brain, the gut, and other tissues. Synuclein is an intrinsically unstable protein prone to misfolding. Synuclein monomers can aggregate to form intermediate oligomers that further assemble into synuclein fibrils that form the Lewy Bodies in PD patients (PMID: 31170426). Chemical, biological, or pharmacological agents that induce changes that mimic PD phenotypes are known to cause synuclein aggregation (PMID: 25497491, PMID: 31265132). Thus, an increase in synuclein aggregation is considered a gold standard assay for studying PD development.

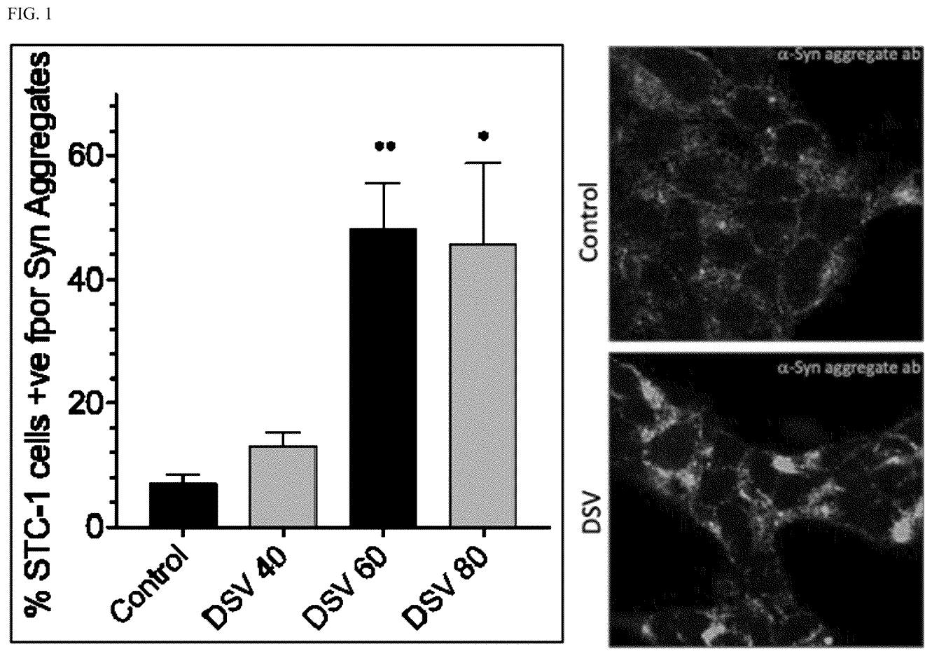

Enteroendocrine cells (STC-1 cells) that are known to constitutively produce and secrete basal levels of synuclein protein (PMID: 28614796) were selected to test the hypothesis that Desulfovibrio may induce aggregation of α-synuclein intestinal cells. STC-1 cells were infected with Desulfovibrio vulgaris (DSV) and the cells were processed for immunofluorescence to visualize synuclein staining using anti-α-synuclein aggregate antibody (abcam, ab209538). DSV induces leaky gut and the inflammatory cascade, each of which is implicated in the development of PD (PMID: 36671785). DSV at a multiplicity of infection (MOI) of 60 and MOI 80 caused a significant increase in perinuclear synuclein aggregates at 24 hours post infection (DSV60: 48.33±7.26 versus control: 7.12±1.39, p<0.01; DSV80: 45.7±13.05 versus control, p<0.05) (FIG. 1). Also, aggregates with lower MOI such as MOI 40 could be visualized at the 48-hour time point.

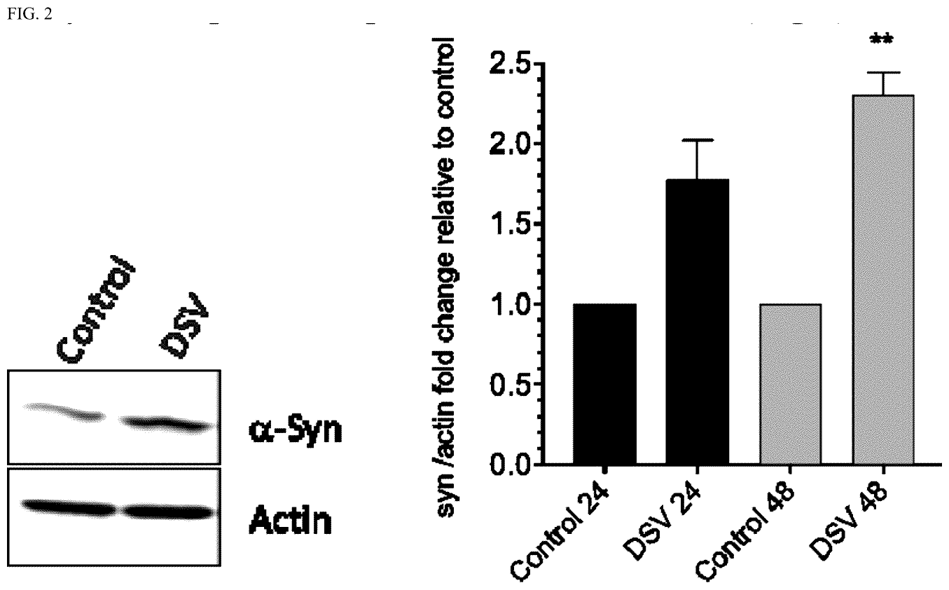

The hypothesis that DSV may increase the expression of α-synuclein by STC-1 cells was also tested. DSV induced synuclein protein expression when compared to control at 24 hours (DSV: 1.77±0.24 versus Control: 1.0) and 48 hours post-infection (DSV: 2.30±0.14 versus Control: 1.0, p<0.01) suggesting that DSV also induces α-synuclein protein expression in STC-1 cells (FIG. 2).

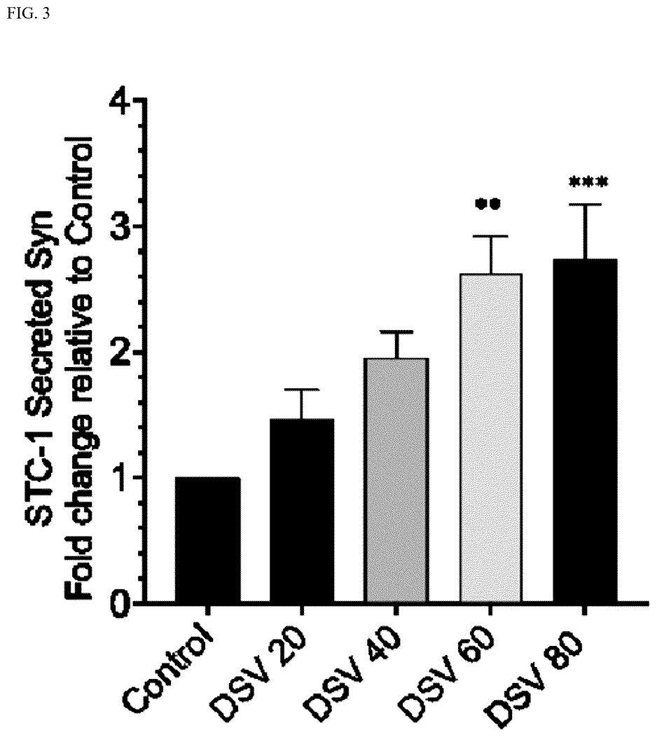

Next, the hypothesis that DSV may increase secretion of α-synuclein by STC-1 cells was tested using ELISA to detect synuclein in the extracellular fluid. There was a dose dependent increase in extracellular synuclein in DSV-treated cells as compared to control uninfected cells. A significant increase was found at higher doses (DSV60: 2.62±0.29 versus control: 1.0, p<0.01; DSV80: 2.73±0.44 versus control, p<0.001) (FIG. 3).

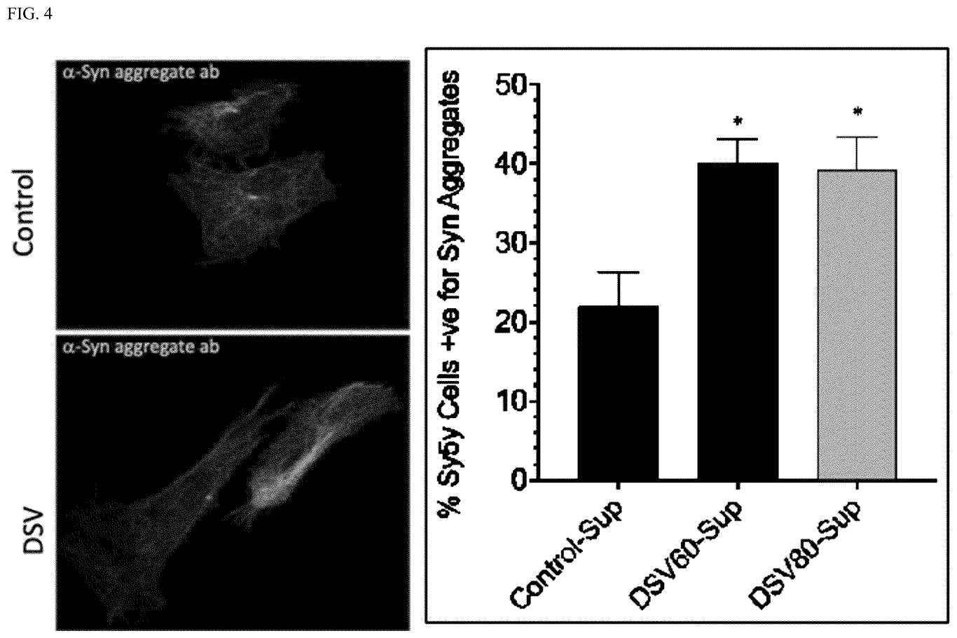

Next, the extent to which DSV-triggered secretion of synuclein from intestinal cells leads to aggregation of synuclein in neurons—i.e., evidence to support a potential mechanism by which gut DSV may induce synuclein aggregates in the intestine which may then be transported to the brain—was investigated. Further, the extent to which supernatant from DSV-infected STC-1 may cause synuclein aggregation in neuronal cells was tested. Sy5y cells, a human neuronal cell line commonly used as an in vitro model to study neurodegenerative diseases (PMID: 36722247), were used. STC-1 cells were infected with DSV for 24 hours. Following infection, the culture supernatants (sup) were collected and passaged through 0.2 μm filters to remove any bacteria and then added to Sy5y cells for 24 hours. DSV-infected STC-1 sup significantly increased the percentage of Sy5y cells positive for synuclein aggregates when compared to sup added from uninfected control STC-1 cells (FIG. 4). Taken together, the findings suggest that DSV induces expression, aggregation, and secretion of synuclein from intestinal cells and extracellular synuclein secreted from DSV-infected STC-1 (among other secreted factors) could also cause synuclein aggregation in neuronal cells. The data support a potential mechanism by which a rare resident gut bacteria DSV can trigger a sequence of events starting from the intestinal cells and ending in neuronal cells that result in the synucleinopathy seen in PD.

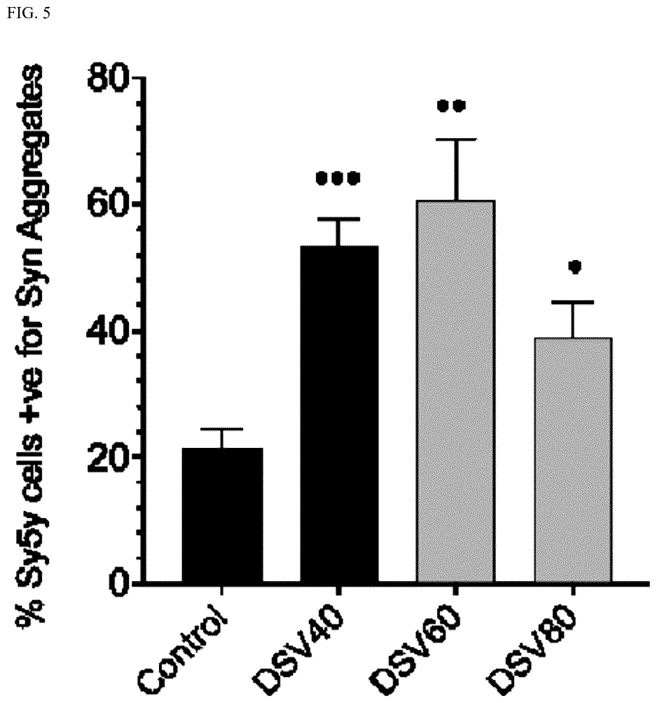

The hypothesis that DSV may infect neurons to increase synuclein aggregation was tested next. Sy5y cells were directly infected with DSV and counted the number of synuclein aggregates within these cells (FIG. 5). This experiment is based on the idea that DSV as gut bacteria can translocate from the gut into CNS in the setting of a leaky gut that allows bacteria to enter the blood stream. DSV can cause leaky gut by increasing Snail transcription factor. It is possible that DSV could also disrupt blood brain barrier by activating Snail since Snail is involved in disrupting the blood brain boundary (PMID: 27588479). In addition, translocation of gut bacteria to the brain occurs via the vagus nerve in the setting of dysbiosis (PMID: 37693595). Thus, it is possible that DSV or its secreted products may directly access neuronal cells.

Sy5y cells were infected for 24 hours with DSV using various MOIS. DSV induced synuclein aggregates in Sy5y cells similar to STC-1 cells (DSV40: 53.46±4.20 versus control: 21.43±2.94, p<0.001; DSV60: 60.57±9.71 versus control, p<0.01, DSV80: 38.92±5.60, p<0.05) (FIG. 5). These findings suggest that DSV directly induce synuclein aggregation in neuronal cell. Taken together, the findings suggest that DSV is capable of causing aggregation of synuclein in both intestinal and neuronal cells and that the aggregation-inducing signals from DSV-infected intestine cells could be further relayed to neuronal cells via extracellular secreted factors even in the absence of DSV (bacteria were filtered out of the supernatant in our study).

### Effect of DSV on Dopamine Pathway

A hallmark feature of PD is the deficiency of the neurotransmitter dopamine and a loss of dopaminergic neurons. Current treatment of PD is directed at administering L-DOPA, a precursor of dopamine. Dopamine (DA) is a catecholamine that is produced by dopaminergic neurons in the substantia nigra of the brain. Dopamine is involved in many physiological functions to include motor control and cognition. Dopamine is synthesized in the cytosol in a two-step process from the amino acid L-tyrosine. In the first step, L-tyrosine is hydroxylated to L-dihydroxyphenylanaline (L-DOPA). This rate limiting reaction is catalyzed by the enzyme tyrosine hydroxylase (TH). In the second step, L-DOPA is converted to dopamine by decarboxylation, a reaction catalyzed by the enzyme aromatic amino acid decarboxylase (AADC). Once synthesized, dopamine is transported into synaptic vesicles by the action of vesicular monoamine transporter-2 (VMAT-2) (PMID: 24548101). Dopamine is then released into synaptic cleft. DA also interacts with dopamine receptors in post-synaptic neurons to exert its physiologic effect. Extracellular DA is also taken up from the synaptic cleft into the presynaptic neuron by dopamine transporter (DAT)(PMID: 23968642).

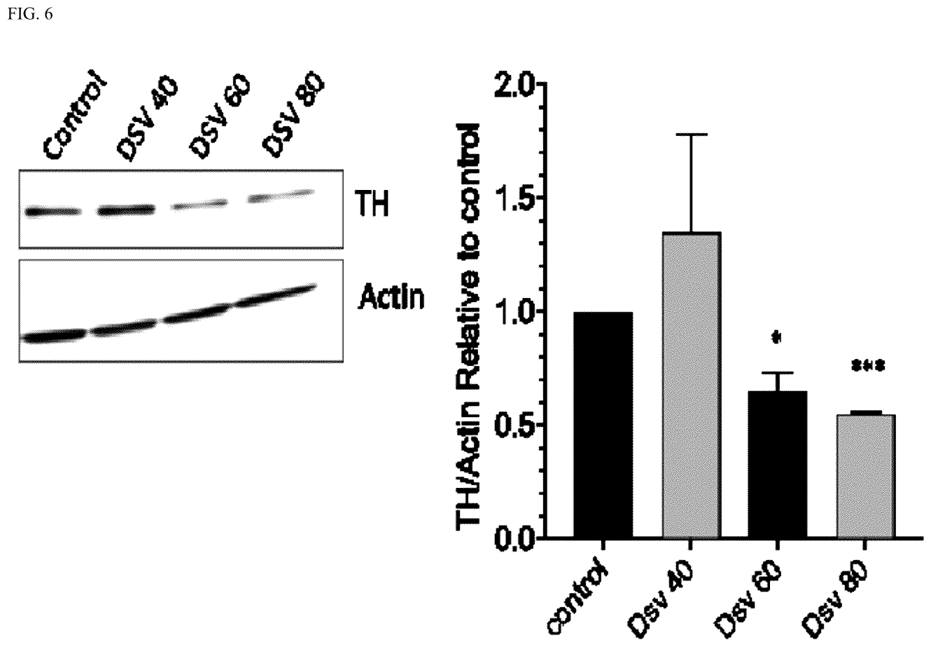

Tyrosine hydroxylase (TH) levels are diminished in PD patients, an enzyme involved in the rate-limiting step in DA biosynthesis. The hypothesis that DSV may inhibit the expression of tyrosine hydroxylase in Sy5y cells was tested. DSV inhibited TH expression in comparison to control untreated Sy5y cells (DSV60: 0.65±0.01 versus control: 1.0, p<0.05; DSV80: 0.59±0.01, p<0.001) (FIG. 6).

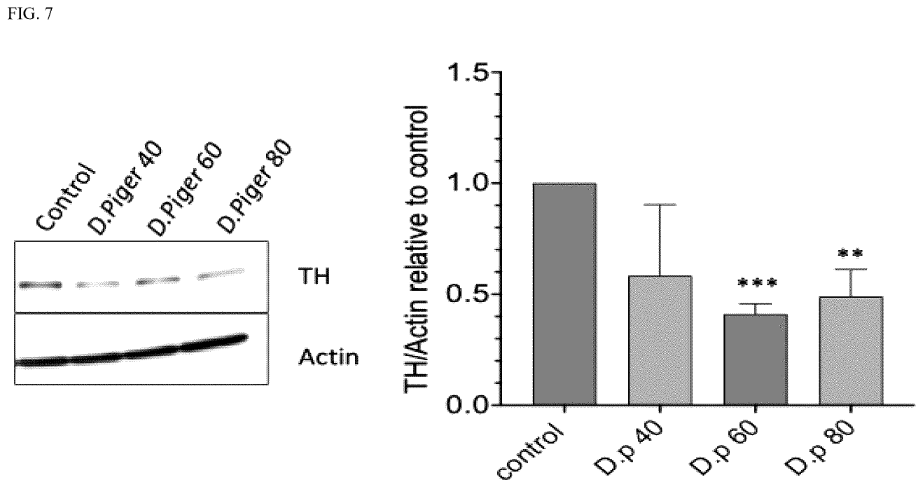

Desulfovibrio piger is a species of sulfate reducing bacteria that is found more prominently in PD patients (PMID: 34012926, PMID: 37197200). Therefore, the hypothesis that D. piger may inhibit the expression TH in Sy5y cells similar to the effects of DSV was tested. Cells were infected with D. piger at MOIs comparable to DSV, for 24 hours. Similar to the effects of DSV, D. piger significantly inhibited TH expression at MOI 60 (DSV: 0.41±0.04 versus control: 1.0, p<0.001) and at MOI 80 DSV: 0.49±0.12, p<0.001) (FIG. 7). These results further confirm findings that sulfate reducing bacteria inhibits TH expression in neuronal cells.

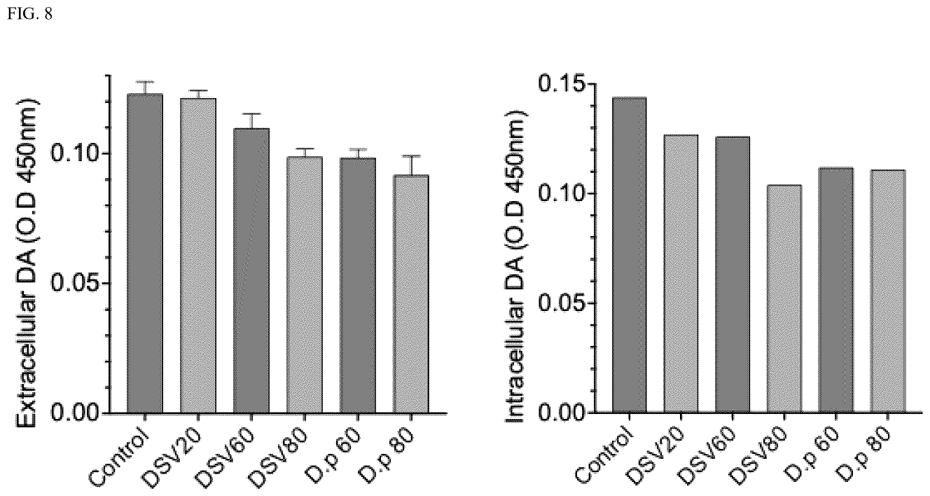

Next, the hypothesis that DSV may inhibit dopamine production in neuronal Sy5y cells was tested. Undifferentiated Sy5y cells (used in this study) produce low levels of dopamine. Both intracellular and extracellular dopamine by ELISA (myBiosource MBS264001) and found a small but dose-dependent decrease in extracellular dopamine quantities in DSV-infected and D. piger-infected cells when compared to uninfected cells (Control). Intracellular dopamine levels were also inhibited in DSV-infected and D. piger-infected cells when compared to uninfected cells (Control) (FIG. 8).

Thus far, the preliminary data explain the link between sulfate reducing bacteria and PD by showing for the first time that sulfate reducing bacteria may contribute to development of PD by 1) inducing synuclein expression and aggregation in intestinal cells, 2) releasing synuclein from intestinal cells, 3) inducing synuclein aggregation in neuronal cells directly or indirectly through exposure to secretions from DSV-infected intestinal cells, 4) inhibiting a dopamine synthesizing enzyme tyrosine hydroxylase in neuronal cells, and/or 5) inhibiting dopamine production and release.

### Proposed Mechanisms of DSV Function in Parkinson's Disease

The data show that DSV increases α-synuclein protein expression and aggregation in intestinal cells. In addition, there is an increased extracellular synuclein from enteroendocrine cells in response to DSV. As synuclein can be transported through vagus nerve and/or through blood stream into the brain and could seed synuclein aggregation in the brain, it is possible that DSV-induced synuclein could travel from GI to the CNS. The in vitro data provided herein support this proposed mechanism. The data show that when conditioned medium from DSV-infected STC-1 enteroendocrine cells was added to neuronal Sy5y cells, it caused increased synuclein aggregation in Sy5y cells. The data also show that DSV affects dopamine synthesis in neuronal cells by inhibiting the rate-limiting protein tyrosine hydroxylase (TH) that carries out the first step of DA synthesis by converting amino acid L-tyrosine to L-DOPA. Both D. vulgaris and PD-associated D. piger inhibit protein expression of TH in Sy5y cells and inhibit dopamine in a dose-dependent manner. The DSV-induced effects observed in the study may be effected through one or more of the following mechanisms.

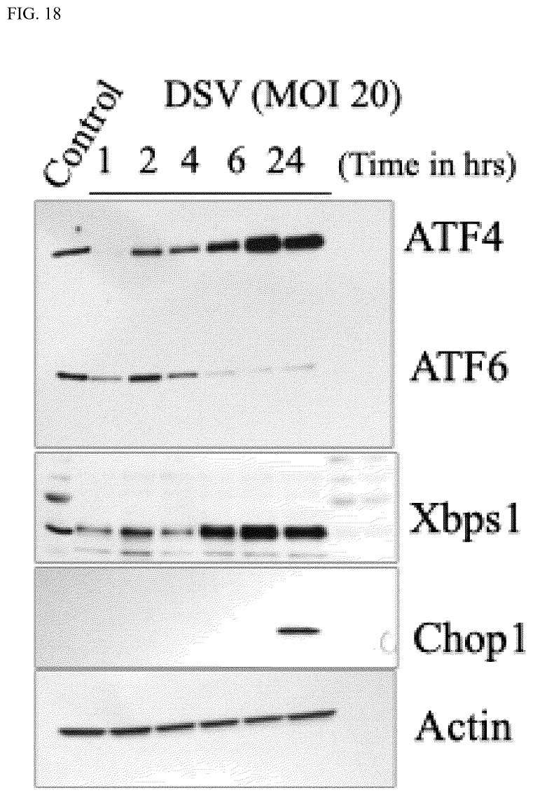

Endoplasmic reticulum (ER) stress. Postmortem brains of PD patients show accumulation of ER stress markers (PMID: 19433866). ER stress occurs as a result of overwhelming the ER leading to a compromised capacity to properly fold protein, resulting in the accumulation of unfolded or misfolded proteins. The data provided herein suggest that DSV causes ER stress. RAW264.7 macrophages were infected with DSV for various time points and the markers for three major arms of ER stress and unfolded protein response (UPR), namely ATF4, ATF6 and XBP1, and downstream C/EBP homologous protein (CHOP) were detected by Western blotting (FIG. 18). Thus, DSV could cause aggregation of synuclein by promoting ER stress.

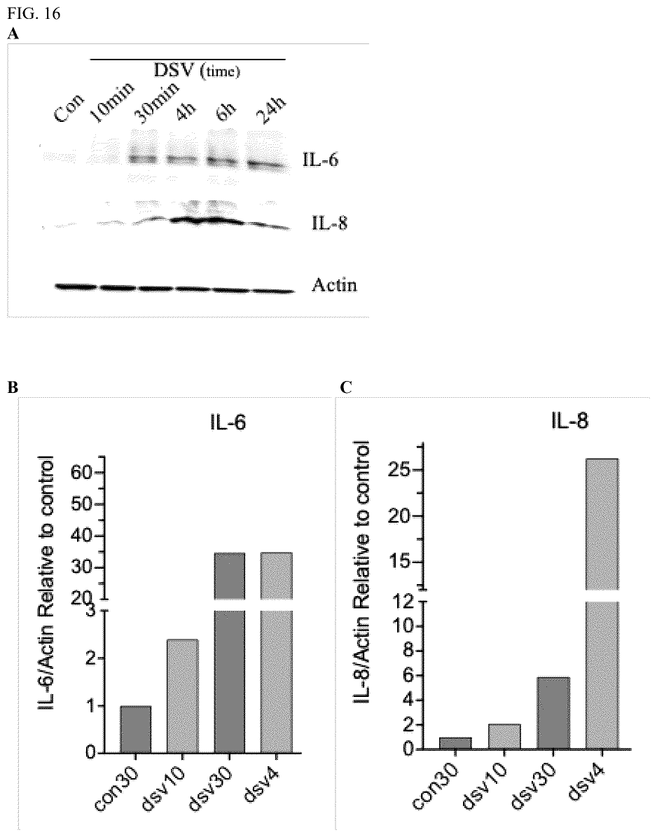

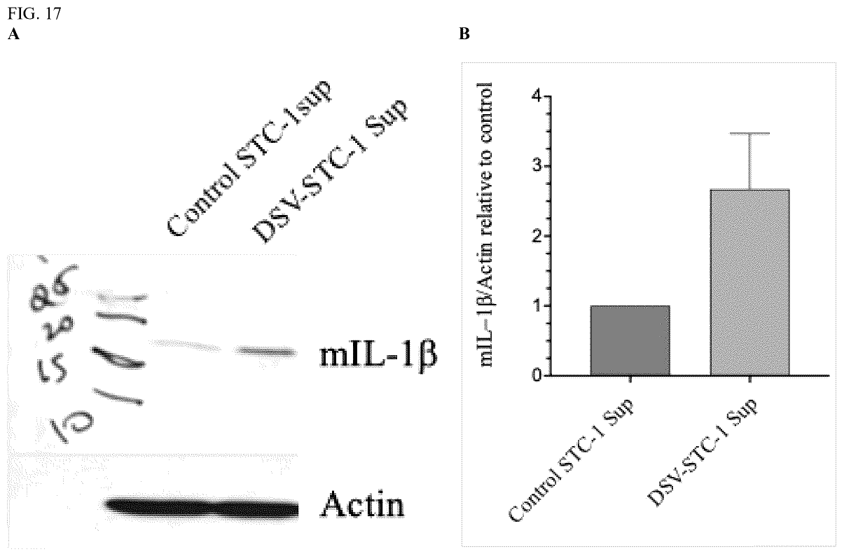

Neuroinflammation. DSV may contribute in neuroinflammation, a hallmark observation in PD. DSV activates the expression and secretion of TNF-α, a proinflammatory cytokine and a known-mediator of neuroinflammation in PD (PMID: 35667491). Additionally, increased levels of TNF-α are found in the brains of PD patients (PMID: 8015728). TNF-α has also been shown to cause synuclein aggregation, secretion, and its cell-to-cell propagation (PMID: 35790884, PMID: 33543132). Thus, it is possible that DSV-induced TNF-α secreted from immune cells, and/or possibly from enteroendocrine cells, is transported to the CNS and may further cause neuroinflammation and synuclein aggregation. DSV could also directly activate TNF-α secretion in glial cells. This disclosure provides data showing that DSV promotes expression of proinflammatory cytokines in HMC3 human microglial cells. Whether DSV induced pro-inflammatory signaling indirectly was tested by adding conditioned medium (CM) from DSV-infected STC-1 intestinal enteroendocrine cells onto HMC3 cells (FIG. 16). Cell lysates were analyzed for the expression of key pro-inflammatory cytokines TNF-α, IL-1β, IL-6, IL-8 as well as downstream phosphorylated STAT3 by Western blotting (FIG. 17).

Inducible nitric oxide synthase (iNOS) expression (PMID: 39338507). The role of iNOS, an enzyme responsible for generating nitric oxide, in neurotoxicity in PD has been suggested in various studies (PMID: 36979000, PMID: 27776473). Higher levels of iNOS have been found in brains of PD patients as well as in animal models (PMID: 18663495). iNOS has also been shown to induce synuclein aggregation (PMID: 22776646). iNOS inhibitors protected against 1-methyl-4-phenyl-1,2,3,6-tetrahydropyridine (MPTP)-induced neurotoxicity and 6-hydroxydopamine (6OHDA)-induced PD-like syndrome in animal studies (PMID: 15949131, PMID: 11778847). Thus, DSV may contribute in PD pathogenesis via its induction of iNOS.

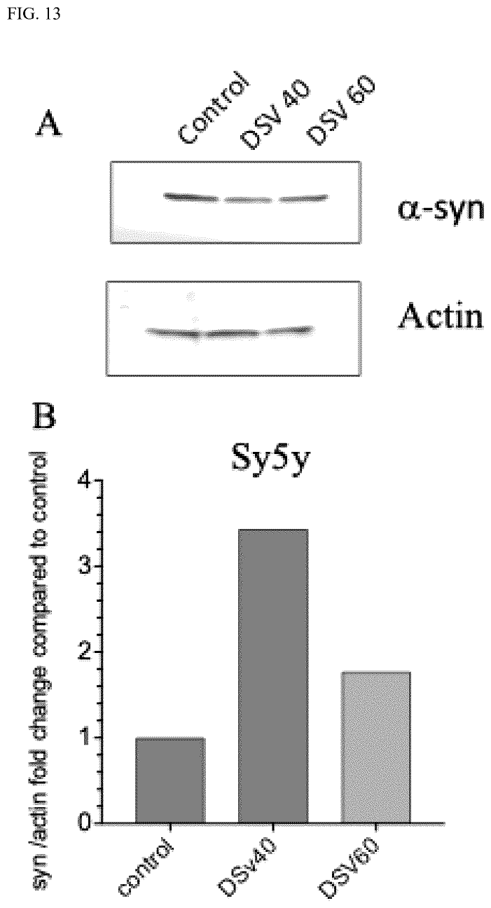

Tyrosine hydroxylase suppression. Alpha-synuclein binds to TH and to suppresses its activity (PMID: 11943812). This disclosure provides data that show that DSV induces synuclein expression in Sy5Y cells (FIG. 13) and concurrently inhibits TH expression in these cells (FIG. 6). Increased synuclein may interact directly with TH, thereby inhibiting its enzymatic activity, providing a potential mechanism by which DSV reduces dopamine synthesis.

Galectin 1/3 balance. DSV may affect the balance of Galectin 1/3, thus tipping the microglia towards chronically activated pro-inflammatory state. Galectins are carbohydrate (glycan) binding proteins that play an active role in inflammation, mediate cell-cell interaction, cellular differentiation, activation of macrophages and microglia and apoptosis (PMID: 40983949, PMID: 26907217). Patients with neurodegenerative diseases experience fluctuating levels of galectins within blood and brain. Galectins Gal-1 and Gal-3 are implicated in PD. Gal-1 modulates microglial activation and provide neuroprotective ability to inhibit neurodegeneration (PMID: 31669519). Gal-3 has a detrimental effect as increased Gal-3 leads to activation of microglia and promotion of neurodegeneration (PMID: 38227529). Cells that are burdened with α-synuclein form Gal-3 puncta due to an increase of ROS and leads to vesicle rupture downstream. The levels of Gal-3 continuously increase as the PD progresses (PMID: 35105290). DSV may cause detrimental effects by suppressing beneficial Gal-1 and activating the harmful Gal-3, leading to microglial activation and promotion of neurodegeneration.

Microglia suppression. DSV may suppress the ability of microglia to ingest and degrade α-syn aggregates, leading to greater neurotoxic effects. Microglia have the innate ability to ingest and degrade alpha-syn aggregates and attenuate their neurotoxic potential (PMID: 18492487). High burden of α-synuclein can overwhelm microglia, impairing their clearance capacity and triggering mitochondrial dysfunction, metabolic stress, and release of pro-inflammatory cytokines, thereby linking aggregate accumulation to neuroinflammation and neuronal stress (PMID: 34555357, PMID: 35599331). DSV may reduce microglial phagocytosis to clear pathogenic aggregates, increasing microglial-induced neuroinflammation, and may facilitate microglial secretion of exosomes containing harmful protein aggregates, which may further lead to α-synuclein propagation (PMID: 38216911, PMID: 40046336).

Astrogliosis. DSV may promote chronic astrogliosis leasing to sustained release of proinflammatory cytokines resulting in loss of DA neurons and PD pathology. Astrogliosis, triggered by oxidative stress and abnormal signals, is a reactive state of astrocytes that is initially protective but becomes detrimental when chronic (PMID: 27241943). The JAK/STAT3 pathway drives astrogliosis, while NF-κB, calcineurin, and MAPK help restore astrocytes to a normal state (PMID: 30617153). Chronic activation leads to sustained pro-inflammatory cytokine release, contributing to dopaminergic neuron loss and worsening Parkinson's disease pathology. (PMID: 30617153). Modulating JAK/STAT3 signaling may therefore represent a therapeutic strategy to reduce neuroinflammation (PMID: 30322407).

### Methods that Block Synuclein Aggregation

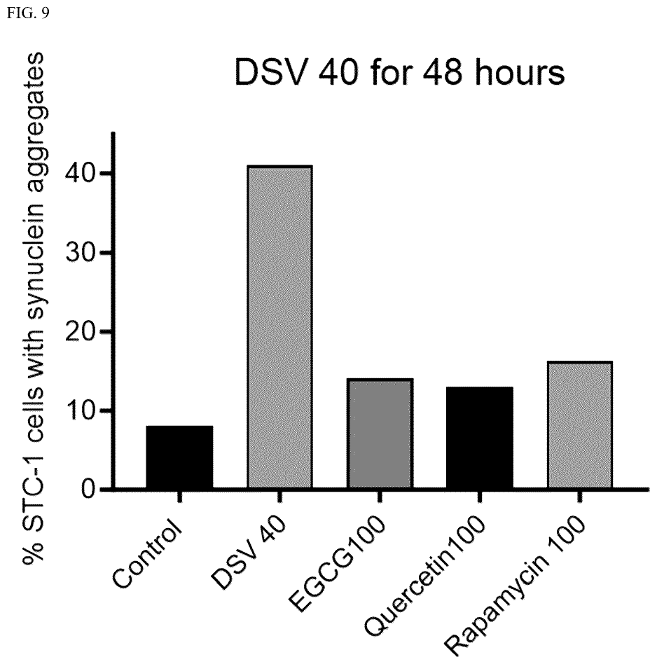

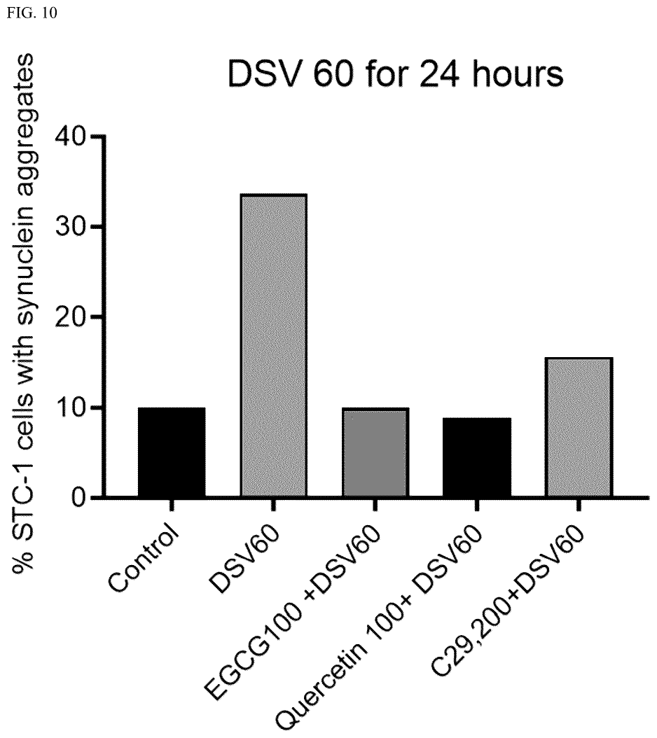

EGCG: Epigallocatechin gallate (EGCG) is a catechin found in green tea. EGCG has been well studied for its protective role against synucleinopathy associated with PD. EGCG acts by remodeling and converting large synuclein fibrils into nontoxic small protein aggregates (PMID: 20385841). In addition, EGCG also prevents synuclein aggregation and fibril formation (PMID: 27364962). These properties of EGCG makes it a promising candidate to test its efficacy in treating synucleinopathies in PD. In this study, the extent to which EGCG could inhibit DSV-induced synuclein aggregation was tested. Pre-treatment of STC-1 cells with 100 μM EGCG for three hours before a DSV challenge for 24 hours or 48 hours inhibited the percentage of cells with DSV-induced aggregates at low MOI DSV40 for 48 hours (DSV: 41% versus DSV+EGCG: 14.1%) as well as at higher MOI DSV60 at 24 hours (DSV: 33.7% versus DSV+EGCG: 10%) (FIG. 9 and FIG. 10).

Quercetin: Quercetin is a flavonoid that naturally occurs in fruits and vegetables and green tea and carries strong antioxidant properties. Quercetin exhibits anti-fibrilization and aggregation effects in synuclein (PMID: 16681381). Additionally, quercetin can inhibit neuroinflammation and also induces autophagy to carry out its neuroprotective effects (PMID: 27756054). Pre-treatment of STC-1 cells with 100 μM quercetin for three hours before DSV challenge for 24 hours or 48 hours inhibited the percentage of cells with DSV-induced synuclein aggregates both at 24 hours (DSV: 33.7% versus DSV+Quercetin: 8.9%) and at 48 hours of DSV infection (DSV:41% versus DSV+Quercetin:13%) (FIG. 9 and FIG. 10).

Rapamycin: Rapamycin is an mTOR (mammalian target of rapamycin) inhibitor involved in inducing autophagy. Rapamycin protects intestinal barrier function by inducing autophagy (PMID: 25616664). Rapamycin has also been tested for its protective roles in neurodegenerative diseases. Rapamycin exhibited anti-aging effects in animal models (PMID: 33037985). Aging is a primary risk factor in the development of neurodegenerative diseases such as PD. Rapamycin attenuates dopaminergic nigrostriatal degeneration in the MPTP model of Parkinson's disease (PMID: 20089925, PMID: 20844148). Moreover, rapamycin also degraded α-synuclein via autophagy (PMID: 12719433). Additional unpublished data also show that rapamycin inhibits DSV-induced barrier permeability by inhibiting Snail. Based on these protective functions of rapamycin in PD, the extent to which rapamycin could inhibit DSV-induced synuclein aggregation in intestinal cells was investigated. FIG. 9 shows that pre-treatment of STC-1 with 100 nM rapamycin for three hours before DSV challenge for 48 hours inhibited the percentage of cells with DSV-induced synuclein aggregates (DSV: 41% versus DSV+RAP 100 nM:16.3%).

TLR2 inhibitor C29: As toll-like receptor 2 (TLR-2) signaling is positively linked to PD, the extent to which inhibiting TLR-2 signaling could alleviate DSV-induced synuclein aggregation in STC-1 cells was tested. C₁₆H₁₅NO₄ (C29) is a TLR-2 inhibitor that blocks TLR2/1 and TLR2/6 signaling in response to TLR-2 agonists (PMID: 25870276). Inhibiting TLR-2 signaling by C29 blocks DSV-induced proinflammatory TNF-α and iNOS production. (PMID: 39338507). In this study, the efficacy of C29 to prevent DSV-induced synuclein aggregates in STC-1 intestinal cells was tested. Cells were pre-treated with C29 at 200 μM for three hours before infection with DSV MOI60 for 24 hours. There was a decrease in the percentage of DSV-infected cells carrying synuclein aggregates in the presence of C29 (DSV: 33.7% versus DSV+C29: 15.7%, FIG. 10), suggesting that TLR-2 signaling is involved in DSV-mediated effects on synuclein and identifies C29 as a potential inhibitor of this pathway.

Intestinal Alkaline Phosphatase (IAP): IAP is an intestinal defense protein that is known for its role in protecting the intestinal epithelial barrier via functions such as LPS detoxification, inhibition of pro-inflammatory cytokines, and induction of autophagy (PMID: 34944428). IAP inhibits DSV-induced increased paracellular permeability (PMID: 35694541). While the role of IAP in ameliorating neurodegenerative diseases has not been explored, IAP decreases neuroinflammation after heart failure (PMID: 34421655). IAP may decrease neuroinflammation by inhibiting DSV-induced intestinal barrier dysfunction and/or by inhibiting proinflammatory cytokines such as TNF-α. IAP could also inhibit synuclein aggregation by inducing autophagy that is responsible for degrading the accumulated synuclein aggregates (PMID: 12719433).

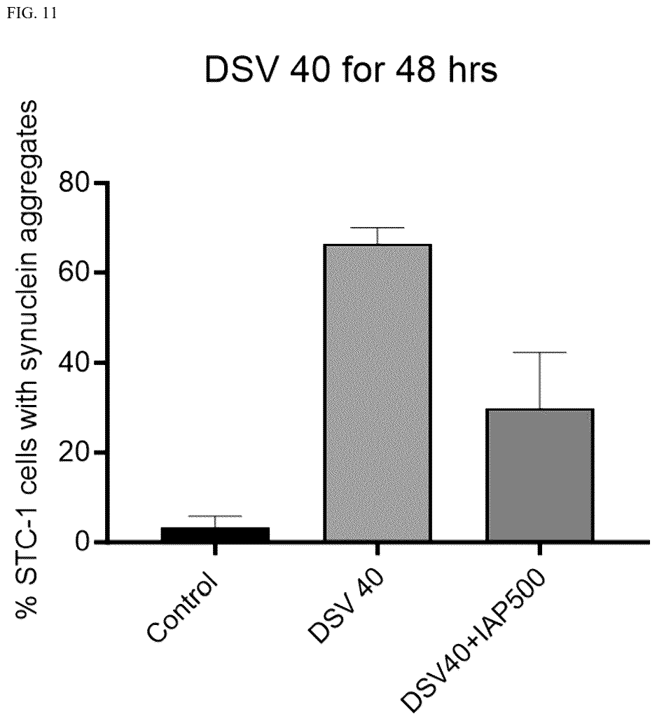

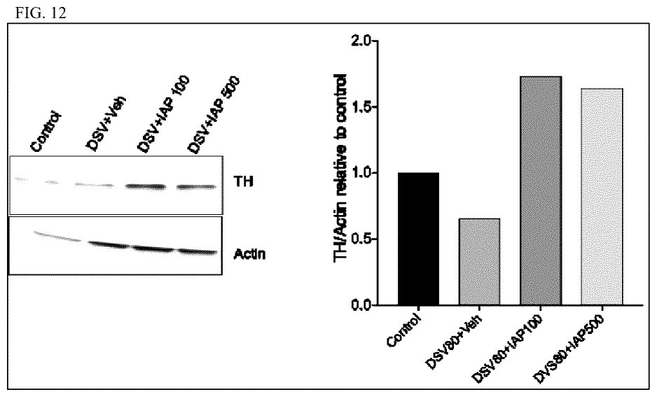

Based on these functions of IAP, the extent to which IAP could inhibit DSV-induced synuclein aggregates was tested. STC-1 cells were first treated with IAP at 500 U/ml or the vehicle control for 24 hours. Cells were then infected with DSV for 48 hours. IAP showed a trend towards inhibition of DSV-induced synuclein aggregates (DSV: 66.40%±3.600 versus IAP+DSV: 29.87%±12.47) (FIG. 11). The extent to which IAP prevented DSV-mediated inhibition of TH expression in neuronal cells also was tested. Pre-treatment of Sy5y cells with IAP inhibited DSV-mediated inhibition of TH expression (Control: 1.00; DSV: 0.65; DSV+IAP100: 1.73; DSV+IAP500: 1.63) (FIG. 12). These findings suggest that IAP could potentially offer a novel therapeutic avenue for inhibiting DSV-induced synuclein aggregation that may alleviate synucleinopathy associated with PD. IAP may also ameliorate dopamine synthesis dysfunction by DSV by upregulating TH expression.

Tissue-nonspecific alkaline phosphatase (TNAP) is produced, for example, by cells in the liver, bones, and kidneys. TNAP may be a suitable alternative to IAP in inhibiting DSV-induced synuclein aggregates.

### Therapeutic Compositions

This disclosure therefore describes compositions a methods for treating Parkinson's Disease in a subject. Generally, the compositions and methods involve administering to the subject a composition that includes at least one component that interferes with a mechanism by which α-synuclein forms aggregates.

In one or more embodiments, the method can include administering to the subject a composition that targets gut dysbiosis characterized by bloom of sulfate reducing bacteria. Exemplary compositions can include, but are not limited to an antibiotic, a prebiotic, a probiotic, a community of bacteriophages, and/or a host defense protein such as, but not limited to, intestinal alkaline phosphatase.

In one or more embodiments, the method can include administering to the subject a composition that suppresses luminal growth of sulfate reducing bacteria and/or binding of its metabolite hydrogen sulfide. Exemplary compositions can include, but are not limited to, MgO, molybdenum, and/or bismuth salts.

In one or more embodiments, the method can include administering to the subject a composition that targets a TLR2 signaling pathway activated by sulfate reducing bacteria. Exemplary compositions can include an anti-TLR2 antibody and/or a TLR2 inhibitors such as, but not limited to, C29, CU-CPT22, robinin, kaempferol-3-O-sophoroside, AN-3485, cholesterol oxidase, Rhodococcus sp, SMU-Y6, MMG-11, and/or phloretin.

In one or more embodiments, the method can include administering to the subject a composition that increases proteasomal or lysosomal destruction of intracellular sulfate reducing bacteria. Exemplary compositions can include an autophagy inducer such as, but not limited to, rapamycin, rapalogs, Torin1, metformin, trehalose, resveratrol, lithium carbonate, carbamazepine, and/or sodium valproate.

In one or more embodiments, the method can include administering to the subject a composition that inhibits synuclein aggregation. Exemplary compositions can include, but are not limited to, intestinal alkaline phosphatase, EGCG, quercetin, and/or an autophagy inducer.

In one or more embodiments, the method can include administering to the subject a composition that inhibits secretion of synuclein or other sulfate-reducing-bacteria-induced factors from intestinal cells. Exemplary compositions can include, but are not limited to, GW4869, cytochalasin B, latrunculin B, caffeine, a cell death inhibitor (e.g., cyclosporin A), a calcium chelator (e.g., BAPTA-AM), and/or an inhibitor of exosome vesicle secretion, exocytosis, and/or tunnel nanotube formation.

In one or more embodiments, the method can include administering to the subject a composition that inhibits redistribution of intracellular dopamine (DA) from synaptic vesicles into the cytosol or that increase VMAT2 protein to facilitate uptake of cytosolic DA into synaptic vesicles. Exemplary compositions include, but are not limited to, PACAP38, desipramine, amoxapine, imipramine, mianserin, pramipexole, apomorphine, and/or bupropion.

In one or more embodiments, the method can include administering to the subject a composition that interferes with sulfate reducing bacteria-induced ER stress. Exemplary compositions can include, but are not limited to, salubrinal, 4-phenyl butyric acid, tauroursodeoxycholic acid (TUDCA), tubercidin, berberine, and/or delphinidin.

In one or more embodiments, the method can include administering to the subject a composition that interferes with sulfate reducing bacteria-induced proinflammatory response. Exemplary compositions include, but are not limited to, simvastatin, α-mangostin, infliximab, an anti-TNF-α antibody, catechins, cannabidiol, curcumin, and/or andrographolide.

In one or more embodiments, the method can include administering to the subject a composition that inhibits sulfate reducing bacteria-induced nitric oxide synthase. Exemplary compositions can include, but are not limited to, agmatine, L-thiocitrulline, tilarginine, aminoguanidine, and/or rheosmin.

In one or more embodiments, the method can include administering to the subject a composition that interferes with inhibition of tyrosine hydroxylase (TH) expression or that increase tyrosine hydroxylase expression. Exemplary compositions can include, but are not limited to, intestinal alkaline phosphatase, estrogen, cAMP, aspirin, and/or glucocorticoids.

In one or more embodiments, the method can include administering to the subject a composition that inhibits binding of synuclein to TH. In one or more of these embodiments, the composition may change the conformation of synuclein so that its affinity for TH is decreased. Exemplary compositions can include, but are not limited to, an anti-synuclein C-terminal antibody. In other embodiments, the composition may stabilize protein-protein interactions of 14-3-3 protein with TH, which leads to its activation. Exemplary compositions can include, but are not limited to, fusicoccin A, and/or Pyrrolidone 1.

In one or more embodiments, the method can include administering to the subject a composition that modulates Gal-1 and/or Gal-3. Exemplary compositions can include one or more inhibitors of Gal-3 and/or one or more activators of Gal-1. Exemplary Gla-3 inhibitors include, but are not limited to, olitigaltin, belapectin, modified citrus pectin (MCP), an anti-Gal-3 neutralizing monoclonal antibody (e.g., those described in PMID: 37652913; PMID: 33580170), and/or Gal-1 supplementation such as, for example, using recombinant Galectin-1 or Gal-1-nanoparticles. Exemplary Gal-1 activators include, but are not limited to, TGF-β, TGF-β (e.g., all-trans retinoic acid (ATRA)), vitamin D3, a TGF-β receptor agonist (e.g., P144, curcumin, thrombospondin 1, tacrolimus), sodium butyrate, and/or retinoic acid.

In one or more embodiments, the method can include administering to the subject a composition that overcomes DSV effects and enhance phagocytosis and clearance of synuclein aggregates by microglia. Exemplary compositions can include a TREM2 agonist, a CD33 inhibitor, a TFEB activator, and/or an NLRP3 inflammasome inhibitor. Triggering Receptor Expressed on Myeloid Cells 2 (TREM2) promotes microglial activation, survival, and phagocytosis, and clearance of α-synuclein aggregates. Exemplary agonists of TREM2 include, but are not limited to, monoclonal antibodies AL002 and 4D9 (PMID: 41301546, PMID: 32154671, PMID: 32579671, PMID: 40165251). CD33 functions as an inhibitory receptor on microglia that suppresses phagocytosis. Elevated CD33 activity can impair microglial clearance of α-synuclein in PD and promote a dysfunctional, pro-inflammatory state. Exemplary CD33 inhibitors include, but are not limited to, an anti-CD33 antibody and/or an anti-CD33 antibody conjugated drugs (e.g., lintuzumab (HuM195), ATLX-1088, etc.), and/or a peptide inhibitor (e.g., IMAD-001). Transcription Factor EB (TFEB) is a master regulator of lysosomal biogenesis and autophagy and enhances the degradation of internalized α-synuclein, and improves phagocytic efficiency. Exemplary TFEB inhibitors include, but are not limited to, trehalose, curcumin, and/or resveratrol. NLRP3 triggers IL-1β release. α-synuclein can activate NLRP3 in microglia, driving chronic neuroinflammation that impairs phagocytosis and contributes to dopaminergic neuron loss. Exemplary NLRP3 inhibitors include, but are not limited to, MCC950.

In one or more embodiments, the method can include administering to the subject a composition that reduces DSV-induced chronic astrogliosis. Exemplary composition can include, but are not limited to, EI-16004, tocilizumab, a JAK/STAT inhibitor, and/or naloxone. Exemplary JAK/STAT inhibitors include, but are not limited to, AZD1480, baricitinib, ruxolitinib, tofacitinib, and/or AG490.

A therapeutic composition can include any one or any combination of two or more active ingredients set forth above. A therapeutic composition may be formulated with a pharmaceutically acceptable carrier to prepare a pharmaceutical composition. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the active agent(s) without causing any undesirable biological effects or interacting in a deleterious manner with any of the other components of the pharmaceutical composition in which it is contained.

A pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

Thus, the active agent(s) may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including, but not limited to, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the [the active agent] into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

### Methods of Treatment

“Treat” or variations thereof refer to reducing, limiting progression, ameliorating, or resolving, to any extent, the symptoms or signs related to a condition. A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is often initiated before a condition manifests in a subject.

Treating a condition can be prophylactic or, alternatively, can be initiated after the subject exhibits one or more symptoms or clinical signs of the condition. Treatment that is prophylactic—e.g., initiated before a subject manifests a symptom or clinical sign of the condition such as, for example, while an infection remains subclinical—is referred to herein as treatment of a subject that is “at risk” of having the condition. As used herein, the term “at risk” refers to a subject that may or may not actually possess the described risk. Thus, for example, a subject “at risk” of infectious condition is a subject present in an area where other individuals have been identified as having the infectious condition and/or is likely to be exposed to the infectious agent even if the subject has not yet manifested any detectable indication of infection by the microbe and regardless of whether the subject may harbor a subclinical amount of the microbe. As another example, a subject “at risk” of a non-infectious condition is a subject possessing one or more risk factors associated with the condition such as, for example, genetic predisposition, ancestry, age, sex, geographical location, lifestyle, or medical history. Treatment may also be continued after symptoms have resolved, for example to delay or reduce the likelihood of recurrence.

Accordingly, a composition can be administered before, during, or after the subject first exhibits a symptom or clinical sign of the condition or, in the case of infectious conditions, before, during, or after the subject first comes in contact with the infectious agent. Treatment initiated before the subject first exhibits a symptom or clinical sign associated with the condition may result in decreasing the likelihood that the subject experiences clinical evidence of the condition compared to a subject to which the composition is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the composition is not administered, and/or completely resolving the condition.

Thus, the method includes administering an effective amount of the composition to a subject having, or at risk of having, a particular condition. In this aspect, an “effective amount” is an amount effective to reduce, limit progression, ameliorate, or resolve, to any extent, a symptom or clinical sign related to the condition.

The amount of the active agent(s) administered can vary depending on various factors including, but not limited to, the specific active agent(s), the extent to which other one or more additional active agent(s) are being co-administered, the weight, physical condition, and/or age of the subject, and/or the route of administration. Thus, the absolute weight of active agent(s) included in a given unit dosage form can vary widely, and depends upon factors such as the species, age, weight, and physical condition of the subject, and/or the method of administration. Accordingly, it is not practical to set forth generally the amount that constitutes an amount of active agent(s) effective for all possible applications. Those of ordinary skill in the art, however, can readily determine the appropriate amount with due consideration of such factors.

For example, certain active agents may be administered at the same dose and frequency for which the active agent has received regulatory approval. In other cases, certain active agent(s) may be administered at the same dose and frequency at which the active agent is being evaluated in clinical or preclinical studies. One can alter the dosages and/or frequency as needed to achieve a desired level of effect for the subject. Thus, one can use standard/known dosing regimens and/or customize dosing as needed.

A single dose may be administered all at once, continuously for a prescribed period of time, or in multiple discrete administrations. When multiple administrations are used, the amount of each administration may be the same or different. For example, a dose of 1 mg per day may be administered as a single administration of 1 mg, continuously over 24 hours, as two or more equal administrations (e.g., two 0.5 mg administrations), or as two or more unequal administrations (e.g., a first administration of 0.75 mg followed by a second administration of 0.25 mg). When multiple administrations are used to deliver a single dose, the interval between administrations may be the same or different.

In one or more embodiments, the active agent may be administered, for example, from a single dose to multiple doses per week, although in one or more embodiments the method can involve a course of treatment that includes administering doses of the active agent at a frequency outside this range. When a course of treatment involves administering multiple doses within a certain period, the amount of each dose may be the same or different. For example, a course of treatment can include an initial loading dose, followed by a maintenance dose that is lower than the loading dose. Also, when multiple doses are used within a certain period, the interval between doses may be the same or be different.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

As used herein, “have,” “has,” “having,” “include,” “includes,” “including,” “comprise,” “comprises,” “comprising” or the like are used in their open-ended inclusive sense, and generally mean “include, but not limited to,” “includes, but not limited to,” or “including, but not limited to.” Further, wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, that which follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

As used herein, the word “exemplary” means to serve as an illustrative example and should not be construed as preferred or advantageous over other embodiments.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

In the preceding description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

In several places throughout the above description, guidance is provided through lists of examples, which examples can be used in various combinations. In each instance, the recited list serves only as a representative group and should not be interpreted as an exclusive list.

### EXAMPLES

The present invention is illustrated by the following examples. It is to be understood that the particular examples, materials, amounts, and procedures are to be interpreted broadly in accordance with the scope and spirit of the invention as set forth herein.

### Cell Culture

Mouse small intestinal enteroendocrine cells (STC-1), Human neuronal cells (SH-Sy5y), human microglial cells HMC3, and mouse macrophage-like cells RAW264 cells were purchased from ATCC (Manassas, VA). STC-1 were grown in DMEM containing 10% FBS (Thermo Fisher Scientific, Inc., Waltham, MA). SH-Sy5y cells were grown in 1:1 mixture of Eagles minimum essential medium and F12 medium with 10% FBS. HMC3 cells were grown in EMEM containing 10% FBS. RAW264.7 were grown in DMEM containing 10% FBS. Cell cultures were incubated at 37° C. in a humidified incubator with 5% CO₂. Cells were plated at a density of 1×10⁶ for experiments with treatments and DSV infection.

### Desulfovibrio vulgaris (DSV) Growth

Desulfovibrio vulgaris Hildenborough (ATCC 29579, Manassas, VA) was grown anaerobically in Hungate tubes using Postgate's organic liquid medium with the following composition: 10.56 mM Na₂SO₄, 13.29 mM MgSO4, 4.12 mM L-Cysteine, 0.4% sodium lactate (60% syrup), 0.4% yeast extract, and 0.5% tryptone. Cultures were grown for 24 hours in 5 ml aliquots at 37° C. Bacteria were counted using Quantom Tx cell counter (Logos Biosystems, Aligned Genetics, Inc., Anyang, South Korea). D. piger (ATCC 29098) was also grown in Hungate tubes in anaerobic conditions in Postgate's medium, similar to DSV. On the day of infection, bacteria were centrifuged at 6000 rpm for five minutes and bacteria in the pellet were resuspended in phosphate buffered saline (PBS). Cells were infected with various multiplicity of infections (MOI) of DSV for 24 hours or 48 hours. Control cells were treated with PBS alone.

### Treatments

Intestinal Alkaline phosphatase (IAP, Sigma Aldrich: A2356) at 500 U/ml was added to STC-1 or SH-SY5Y cells for 24 hours prior to adding DSV for 24 hours (for SY5Y) or 48 hours (for STC-1). Vehicle for the control cells was the solution in which IAP was supplied

Epigallocatechin-3-gallate (EGCG, Thermo Fisher Scientific, Inc., Waltham, MA: 449010500, resuspended in DMSO) was added to STC-1 cells at 100 μM for three hours prior to adding DSV for either 24 hours or 48 hours.

Quercetin (Sigma Aldrich: Q4951, resuspended in DMSO) was added to STC-1 cells at 100 μM for three hours prior to adding DSV for either 24 hours or 48 hours.

Rapamycin (Cell Signaling Technology, Danvers, MA: 9904S, resuspended in DMSO) was added to STC-1 cells at 100 nM for three hours prior to adding DSV for either 24 hours or 48 hours.

C29 (InvivoGen, San Diego, CA: TL2-C29, resuspended in DMSO) was added to STC-1 cells at 200 μM three hours prior to adding DSV for 24 hours.

### Treatment of SY5Y and HMC3 Cells With DSV-Infected Intestinal Cell Supernatant

1×10⁶ STC-1 cells were plated in a six-well plate. On the following day, cells were infected with DSV (60 MOI or 80 MOI) for 24 hours. The next day, conditioned medium from control or DSV-infected STC-1 cells were collected and passaged through 0.2 μm filters and added to SY5Y cells, for another 24 hours. SY5Y medium was removed and replaced completely with STC-1 conditioned medium. SY5Y cells were then fixed and processed for immunofluorescence using α-synuclein aggregate antibody. Percentage of cells positive for aggregates were counted and values were compared to cells treated with control uninfected sup from STC-1 cells. Similarly, HMC3 cells grown on a six-well plate were treated with control of DSV-infected conditioned medium (sup) for 30 minutes after which cells were lysed and protein lysates were analyzed by Western blotting.

### Western Blot

Cells were lysed in lysis buffer in the presence of protease and phosphatase inhibitors (Thermo Fisher Scientific, Inc., Waltham, MA). Briefly, cell lysis was carried out for 20 minutes on ice. Lysates were centrifuged at 12,000 rpm for five minutes at 4° C. and supernatants collected and protein concentration determined with Bradford reagent (Thermo Fisher Scientific, Inc., Waltham, MA). 50μg protein samples were run on SDS-PAGE (4-20% tris-glycine) and transferred to nitrocellulose membranes. Membranes were blocked in 5% milk in PBS-T (0.1% Tween 20) for 30 minutes, followed by overnight incubation in antibodies against actin (Cell Signaling Technology, Danvers, MA: 4970) and α-synuclein (Cell Signaling Technology, Danvers, MA: 2642), and tyrosine hydroxylase (Cell Signaling Technology, Danvers, MA: 58844). For pro-inflammatory cytokine expression, antibodies used were TNF-α, IL-1β, IL-6, and IL-8. For ER stress response antibodies used were ATF4. ATF6, Xbps1, and Chop1 (Cell Signaling Technology, Danvers, MA). Antibodies were diluted as recommended by the manufacturer. Blots were incubated with secondary antibodies (Cell Signaling Technology, Danvers, MA: 7074) at room temperature for one hour and developed using enhanced Chemiluminescence HRP signal (Thermo Fisher Scientific, Inc., Waltham, MA). Images were quantified using Image J software

### Immunofluorescence

Cells were grown on coverslips in six-well plates. After treatment, cells were fixed with 4% paraformaldehyde for 15 minutes and washed with PBS three times for five minutes each. Cells were incubated in a blocking solution consisting of 5% FBS and 0.3% Triton-X 100 for one hour. This was followed by overnight incubation with anti-α-syn aggregate-specific antibody (Abcam, Cambridge, UK: ab209538) at 4° C. Cells were then washed with PBS followed by incubation with Alexa fluor 488-labeled secondary anti-rabbit antibody (Thermo Fisher Scientific, Inc., Waltham, MA) for two hours at room temperature. Imaging was done with confocal microscope (FLUOVIEW FV1200, Olympus Corp., Tokyo, Japan). Aggregates were identified manually in Z-projected images using a defined size threshold (>1 μm in diameter). Cells were scored positive if they contained one or more aggregates exceeding this size. Counting was performed consistently across all controls and treatment groups, and at least ˜150-225 cells per condition per experiment were analyzed across minimum of three independent times. The percentage of positive cells was used for statistical comparisons.

### Enzyme-Linked Immunosorbent Assay (ELISA)

Alpha-synuclein level was measured in the filtered STC-1 culture supernatant collected from uninfected or DSV-infected cells using a mouse α-syn ELISA kit (Abcam, Cambridge, UK: ab282865) using the manufacturer's protocol. For dopamine analysis, SH-Sy5y cells were infected for 24 hours with DSV and D. piger at various MOIS. Culture supernatants were collected and cells were harvested and lysed with lysis buffer containing protease and phosphatase inhibitors. The amount of extracellular and intracellular dopamine (in 25 mg lysates) was measured using a double sandwich ELISA kit (myBiosource, San Diego, CA: MBS 264001). OD values were measured at 450 nm.

### Statistical Analysis

All graphs were generated using PRISM 9 (GraphPad Software, Inc., San Diego, CA). Data represents means±SEM from three independent experiments; each performed with three biological replicates pooled per condition per experiment. Fold change differences were represented as values normalized to control (set as 1). Students t-test was used for statistical analysis for all the figures. P values<0.05 were considered statistically significant.

For any method disclosed herein that includes discrete steps, the steps may be performed in any feasible order. And, as appropriate, any combination of two or more steps may be performed simultaneously.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method of inhibiting synuclein aggregation in a subject, the method comprising administering to the subject a composition comprising at least one antibiotic, prebiotic, probiotic, TLR2 antagonist, autophagy inducer, host defense protein, or a combination thereof.

2. The method of claim 1, wherein the composition comprises intestinal alkaline phosphatase, quercetin, epigallocatechin-3-gallate (EGCG), rapamycin, or a combination thereof.

3. A method of inhibiting secretion of synuclein from intestinal cells in a subject, the method comprising administering to the subject a composition comprising GW4869, cytochalasin B, latrunculin B, an inhibitor of exosome vesicle secretion, an inhibitor of exocytosis, an inhibitor of tunnel nanotube formation, caffeine, a cell death inhibitor, a calcium chelator, or any combination thereof.

4. The method of claim 3, wherein the cell death inhibitor is cyclosporin A.

5. The method of claim 3, wherein the calcium chelator is BAPTA-AM.
